(19) United States
(12) Patent Application Publication
(10) Pub. No.: US 2022/0163537 A1
(43) Pub. Date: May 26, 2022

(54) Detection and Quantification of Molecular Species

(71) Applicant: Fast BT UG, Mannheim (DE)

(72) Inventor: Balendu Avvaru, Mannheim (DE)

(21) Appl. No.: 17/598,418

(22) PCT Filed: Mar. 26, 2020
(86) PCT No.: PCT/EP2020/058626

§ 371 Date: Sep. 27, 2021

(30) Foreign Application Priority Data
Mar. 26, 2019 (EP) 19165356.7

(51) Int. Cl. G01N 33/68

(52) U.S. Cl. CPC G01N 33/6887; G01N 2650/00; G01N 2333/4712

(57) ABSTRACT

The invention relates to a conjugate comprising a binding element and a self-polymerising biopolymer. The invention also relates to a kit comprising the conjugate and self-polymerising biopolymer subunits. The invention also relates to a method of using the conjugate to measure the concentration of a target substance comprising binding the conjugate to the substance, isolating the bound conjugate, polymerising biopolymer filament, and calculating the concentration of the target substance.

### FIELD OF THE INVENTION

The present invention relates to a conjugate and also a kit comprising the conjugate. In addition, the invention relates to a method for measuring the amount of a target substance.

### BACKGROUND OF THE INVENTION

There are many circumstances in which it is desirable to determine the presence and quantity of a target substance in a sample. For example, it is often desirable to determine the presence and quantity of pathological microorganisms in samples taken from patients or in the environment such as within hospitals and the like. Further examples include the detection of microorganisms in soil and measuring the amount of growth of those micro-organisms over time.

Known means for detecting and quantifying the amounts of a target substance have the issue that at low concentrations of substance to be detected or quantified, the determination of presence or the calculated concentration or amount tends to be unreliable.

Enzyme-Linked ImmunoSorbent Assay (ELISA) produces a coloured pigment, the amount of which determines the saturation of the apparent colour in the test. The amount of pigment produced is dependent on the amount of enzyme that manages to bind to the substance to be measured. As such, the amount of pigment produced is proportional to the amount of substance to be measured. This is useful because within a given range one would expect that if the concentration of substance were doubled then the colour from the ELISA test would be twice as saturated.

However, techniques like ELISA are less effective when measuring samples having very low concentrations of the measured substance. The relatively low signal-to-noise ratio means that it becomes difficult to determine whether a very low value of colour saturation results from a corresponding very low concentration of the measured substance or simply from measurement error.

It is desirable to provide a means and a method for detecting and quantifying low concentrations of a substance to be measured.

### SUMMARY OF THE INVENTION

According to a first aspect of the invention, there is provided a conjugate for measuring the amount of a target substance comprising:

a. a binding element capable of binding to the substance

b. a self-polymerising biopolymer or a nucleating protein.

The binding element permits that the conjugate can bind to the target substance such that the conjugate becomes linked to the target substance. This causes the self-polymerising biopolymer to become linked to the target substance.

Advantageously the binding element is an antibody. Antibodies permit specific binding to a particular epitope on an antigen. This ensures that it is possible to measure the concentration of a specific target substance even when the target substance is present among similar substances. An antibody can be selected that only binds the specific target so that the self-polymerising biopolymer only becomes linked to the specific target.

Binding elements may be less specific if desired. For example it may be preferable to choose a protein that will generally bind to proteins, sugars, bacterial cell walls, or some other broad target. For example, this could allow one to determine the concentration of sugars in a sample.

Preferably, the nucleating protein is VopF, VopL, an Arp2/3 complex, or formin.

Preferably, the biopolymer is in the form of a self-polymerising biopolymer nucleus for seeding biopolymer polymerisation. Biopolymers such as actin and tubulin cannot begin assembling into polymers (polymerisation) directly from monomers without first forming a nucleus comprising a cluster of monomer units (nucleation). Nucleation is typically the rate determining step, and as such, isolated monomers of these biopolymers are not likely to spontaneously polymerise. By including a nucleus in the conjugate, polymerisation can only begin initially from these conjugates.

Preferably, the biopolymer is actin or tubulin. Actin and tubulin are preferred as they exhibit polymerisation at consistent rates and are capable of geometric acceleration of polymerisation due to the fragmentation of filament strands. Actin and tubulin are self-polymerising and do not require outside enzymes in order for filaments to be extended. Actin and tubulin also form homo-polymers (i.e. polymers consisting of a single type of monomer subunit) and do not require any form of template for polymerisation. This simplifies the reaction by reducing the number of components necessary.

Advantageously, the conjugate further comprises a nanoparticle to which the binding element and the biopolymer are linked. Linking the conjugate via a nanoparticle means it will be easier to attach any further elements to the conjugate such as further binding elements for other targets, or sugar residues to change the properties of the nanoparticle. It also means that properties of the nanoparticle can be used to modify the overall properties of the conjugate. For example, the nanoparticle could be magnetised in order to permit manipulation of the particle, the conjugate elements, and any bound substance with magnetic means.

According to a second aspect of the invention, there is provided a kit comprising:

a. the conjugate of the invention;

b. a plurality of self-polymerising biopolymer subunits.

The conjugate can bind to the target substance therefore linking the self-polymerising biopolymer to the target substance as described above. The plurality of self-polymerising biopolymer subunits once mixed with the conjugate may then begin polymerising from the conjugate.

Preferably, the kit further comprises an agent to prevent spontaneous nucleation or polymerisation of the biopolymer subunits. In suitable conditions, self-polymerising biopolymers may spontaneously begin polymerising before this is intended. Agents such as thymosin beta 4 (Tβ4) prevent spontaneous nucleation of G-actin, thereby preventing monomeric G-actin from spontaneously polymerising.

Preferably, the self-polymerising biopolymer subunits comprise labelled self-polymerising biopolymer subunits. Labelled biopolymer subunits can be added in addition or in place of unlabelled biopolymer. By adding an experimentally detectable label to the biopolymer, it is possible to detect the polymerisation and depolymerisation of the biopolymer.

Preferably, this label is a fluorescent label that will fluoresce in response to excitation from light. Examples of fluorescently-labelled biopolymers include GFP-actin (Green Fluorescent Protein), pyrenyl-actin, NBD-actin (7-chloro-4-nitrobenzeno-2-oxa-1,3-diazole), and SiR-actin (Silicon Rhodamine). Alternatively, the label can be any other form of molecular label such as a radioactive label or a phosphorescent label.

According to a third aspect of the invention, there is provided a method for measuring the amount of a target substance, the method comprising:

a. providing a plurality of conjugates of the invention;

b. binding the conjugates to the target substance;

c. isolating bound conjugates

d. polymerising biopolymer filaments

e. calculating an amount of the target substance.

The method of the invention allows one to use the conjugate of the invention to measure the amount of a target substance.

Preferably, the method further comprises the additional step:
d2. measuring a time value indicative of the time taken for polymerisation and depolymerisation of the biopolymer to reach steady state.

The polymerisation and depolymerisation of the biopolymer will reach steady state more quickly the greater the concentration of bound conjugates in the sample, and vice versa. By measuring the time taken, one can determine an indication of the amount or concentration of the target substance.

Preferably the method further comprises the additional step:
d3. using the time value to calculate a number or concentration of the bound conjugates.

As the time taken to reach steady state is dependent on the number of bound conjugates in the sample, once this time value is determined, one can calculate the number or concentration of bound conjugates in the sample to be a particular value.

Advantageously, calculation in step e comprises using the calculated number of bound conjugates to calculate the amount of the target substance.

Once a value for the number or concentration of bound conjugates has been determined, this value can be related to the concentration of the target substance based on the level of binding of the conjugate to the target substance.

Preferably, step d further comprises increasing the rate at which biopolymer filaments undergo fragmentation, preferably by using sonication.

Increasing fragmentation of biopolymer filaments allows the number of free-ends of filament to increase more rapidly and therefore causes the overall rate of polymerisation in the system to increase more rapidly as well. This allows the reaction to be completed in less time as the polymerisation and depolymerisation reach steady state more quickly. Also the sonication causes the filaments to fragment at more regular lengths, which causes a more regular geometric increase in the growth of the overall polymerisation rate of the system. This allows the reaction to be more readily modelled.

Preferably, a concentration of the bound conjugates is calculated using the following equation:

$$y \vert 0 \vert = A \frac{{e}^{\frac{- k A}{m} {t}_{1 / 2}}}{1 + {e}^{\frac{- k A}{m} {t}_{1 / 2}}}$$

where y|0| is the concentration of bound conjugates at time zero,
where A is the total concentration of biopolymer minus the critical concentration of biopolymer,
where k is the sum of rate constants of biopolymer subunit addition to filament ends,
where m is the average length of the biopolymer filaments
and where t_{1/2} is the time taken to reach the biopolymer concentration that is half of the biopolymer concentration found at steady state.

The regular fragmentation provided by sonication leads to a reaction that can be readily modelled using the equation above. By using the halfway point between the start of the reaction and the time taken to reach steady state, one can make use of a high signal-to-noise ratio data point to characterise the progress of the reaction. With the reaction adequately modelled, it is possible to calculate the concentration of bound conjugates with a high degree of precision. From this concentration of bound conjugates, the concentration of the target substance can be determined based either on experimentation using known concentrations of the target substance or by calculating a predicted value for the number of conjugate binding sites on the target substance.

Advantageously, the polymerisation state of the biopolymer is measured by using fluorescently-labelled biopolymer.

Fluorescently-labelled biopolymers show fluorescence when in the form of filaments. The progress of the reaction towards steady state can be measured through the level of fluorescent light emitted in response to excitatory light provided to the sample.

Advantageously, the polymerisation state of the biopolymer is measured by the level of light scattering caused by the biopolymer filaments.

Biopolymer filaments in solution scatter light passing through them. The greater the extent of polymerisation and therefore the number of filaments in solution, the greater the level of light scattering. Thus light scattering can be measured to determine the extent of polymerisation and determine when the reaction reaches steady state.

Advantageously, the polymerisation state of the biopolymer is measured by viscometry. The polymerisation of biopolymer causes the solution to become more viscous and so viscosity can be used as an indicator of the level of polymerisation.

Advantageously, the polymerisation state of the biopolymer is measured by flow birefringence. Polymerisation of biopolymers affects the birefringence of a sample, due to the alignment of filaments with lateral flow of the solution. This property can be measured through the measurement of the refraction of polarized light in the sample.

Advantageously, the polymerisation state of the biopolymer is measured by providing monomers labelled with donor and acceptor fluorophores. When monomers carrying complementary fluorophores are co-polymerized, this results in fluorescence energy transfer (FRET). The transfer of energy from donors to acceptors occurs predominantly in the filaments and so the measurement of FRET corresponds to the level of polymerisation in the sample.

Advantageously, the polymerisation state of the biopolymer is measured by the difference in ultraviolet absorption spectrum between filaments and monomers

Advantageously, the polymerisation state of the biopolymer is measured by ultracentrifugation. Filaments have higher sedimentation coefficient than monomers and so measurement of the sedimentation coefficient is indicative of the level of polymerisation.

Advantageously, the polymerisation state of the biopolymer is measured by filtration by altering the pore-size of the filtration-media. Alternatively, the polymerisation state of the biopolymer is measured by filtering out the biopolymer filaments with a filtration medium which separates biopolymer filaments. Filaments and subunits differ in their physical sizes and so the proportion of biopolymer passing through or being retained by the filter is indicative of the level of polymerisation.

### BRIEF DESCRIPTION OF THE FIGURES

FIG. 1 shows a schematic representation of a conjugate according to a first embodiment of the invention.

FIG. 2 shows a schematic representation of a conjugate according to a second embodiment of the invention.

FIG. 3 shows a schematic representation of a conjugate according to the first embodiment of the invention in the process of polymerising further actin monomers.

FIG. 4 shows a schematic representation of a conjugate according to the first embodiment having an extended biopolymer filament where the filament is undergoing fragmentation.

FIG. 5 shows a schematic representation of a conjugate and a split-off filament in the process of polymerising further actin monomers.

FIG. 6 shows a schematic representation of an actin filament in steady state demonstrating the “tread-milling” effect.

FIG. 7 shows a graph representing the rate of change of rate of polymerisation of actin filaments in a system.

FIG. 8 shows a graph representing the rate of change of rate of polymerisation of actin filaments in two systems with different bound conjugate concentrations.

### DETAILED DESCRIPTION

Actin is a family of globular multi-functional proteins that form microfilaments. Actin is found in cells as a free monomer called globular-action (G-actin) or as part of a linear polymer microfilament called filamentous-actin (F-actin).

G-actin will not spontaneously polymerise directly into F-actin without first forming a nucleus. Instead, a nucleus of G-actin must first form, to which further G-actin monomers can then bind to form a polymer strand. G-actin has a “minus” pointed end and a “plus” barbed end. In nature, polymerisation proceeds either by the association of a pointed end of a G-actin monomer with the G-actin subunit at the barbed end of an F-actin filament or alternatively by the association of a barbed end of a G-actin monomer with the G-actin subunit at the barbed end of an F-actin filament. Polymerisation then progresses from both the minus and the plus ends along the growing strand. The spontaneous formation of an actin nucleus has a high activation energy and so in nature requires the presence of nucleating factors such as the Arp2/3 complex in order to form. The Arp2/3 complex mimics a G-actin dimer in order to stimulate the nucleation (or formation of the first trimer) of monomeric G-actin.

The growth of actin filaments can be regulated by thymosin and profilin. Thymosin binds to G-actin to buffer the polymerisation process. Profilin binds to G-actin to exchange ADP (Adenosine Di-Phosphate) for ATP (Adenosine Tri-Phosphate) promoting the monomeric addition to the barbed “plus” end of the polymer. Furthermore, the protein thymosin beta 4 (Tβ4) inhibits spontaneous nucleation by sequestering G-actin. Wiskott-Aldrich syndrome homology region 2 (WH2) motifs can modulate actin polymerization and prevent nucleation. Proteins like gelsolin and VopF/L display severing activity on filaments. Capping proteins like CapZ and gelsolin cap the barbed ends, while tropomodulin caps the pointed end. Capping of the ends with these capping proteins prevents the addition of monomers to the respective end.

Proteins like VopF/L, Arp2/3 complex, and formins could act as nucleators that assemble actin-nuclei from G-actin monomers. These nucleating-proteins could also be integrated into the conjugate.

Unlike most polymers, such as DNA, whose constituent monomers are bound together with covalent bonds, the monomers of actin filaments are assembled by weaker bonds. The weak bonds give the advantage that the filament ends can easily release or incorporate monomers. This means that the filaments can be rapidly remodelled.

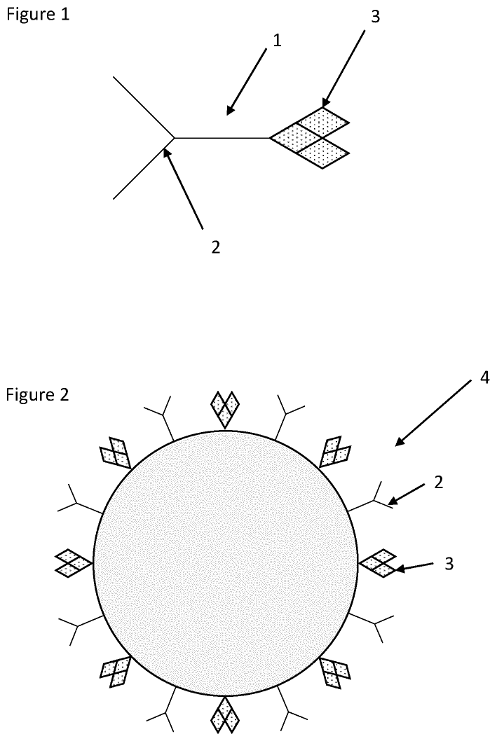

FIG. 1 shows a schematic representation of a conjugate 1 according to a first embodiment of the invention. On a first end of the conjugate 1 is an antibody 2. This antibody 2 is capable of specific binding to a pre-determined target at a first epitope thereof. Covalently linked to this antibody is an actin nucleus 3. It is to be understand that a non-covalent linkage could alternatively be used.

Because the conjugate 1 contains an actin nucleus 3, monomeric G-actin can spontaneously polymerise from this nucleus 3.

The actin nucleus is in the form of spectrin-actin seeds which only present a plus end of the actin for polymerisation while suppressing polymerisation from the minus end of the actin nucleus. This ensures that polymerisation can only occur from a single end of the actin nucleus which ensures that the polymerisation kinetics are more easily modelled.

FIG. 2 shows a schematic representation of a conjugate 4 according to a second embodiment of the invention. The embodiment comprises a nanoparticle to which the antibody 2 and the actin nucleus 3 are bound. This embodiment illustrates that it is not particularly important how the antibody 2 and the actin nucleus 3 are attached to one another in the conjugate 1, simply that they are physically bound.

Use of the conjugate 1 of the first embodiment will now be described. A sample (not shown) containing a target substance or antigen 5 of unknown quantity is provided. An excess of the conjugate 1 is added to the sample at a concentration of between 1 nanomolar and 1 micromolar and mixed thoroughly, for example, by agitation. Subsequently, a plurality of magnetic beads, each linked to an antibody specific for a second epitope of the antigen 5 is provided and the plurality of magnetic beads is added to the sample. It is to be appreciated that the antibody 2 which is specific for the first epitope of the antigen 5 and the antibody linked to the magnetic bead which is specific for the second antibody of the antigen 5 are selected such that the respective antibodies can both bind to the antigen 5 without competing for binding thereof such that when both antibodies are bound to the target antigen 5, the antigen forms a link or “bridge” between the magnetic bead and the actin nucleus 3. It is also to be appreciated that one could use the same antibody for both the conjugate and the magnetic beads provided that the target substance displays multiple copies of the epitope.

The magnetic bead and the components linked thereto are then withdrawn from the sample via magnetic means and are subjected to washing, such as with water or phosphate buffer saline, so as to remove all components except for the conjugate 1. It is to be understood that the amount of the separated conjugate 1 that remains at this stage in the process is equivalent to the amount of the target substance or antigen 5 that was present in the original sample. Profilin, Tβ4, ATP and salts are added to the conjugate 1 to create a polymerisation solution. In addition, G-actin 6 is added to the polymerisation solution, 5% of which is labelled with a fluorescent label thus being fluorescently labelled G-actin 7.

Alternatively or in addition, other means for isolating the bound conjugates could be used such as centrifugation, to centrifugally separate out larger particles for example bacteria with bound conjugate on their surface. Alternatively or in addition, ultra-filtration could be used to the same effect.

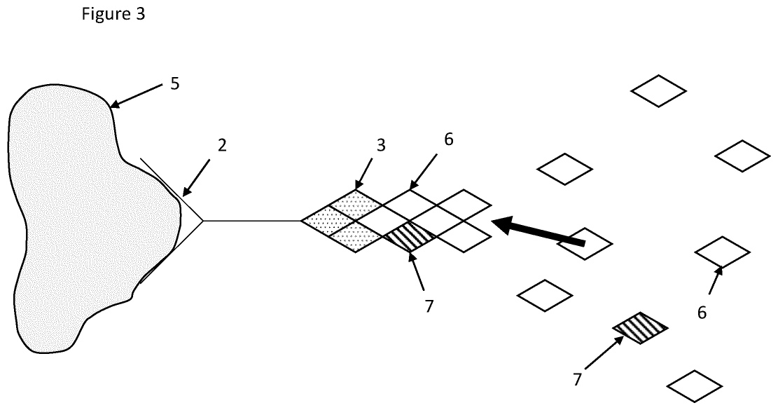

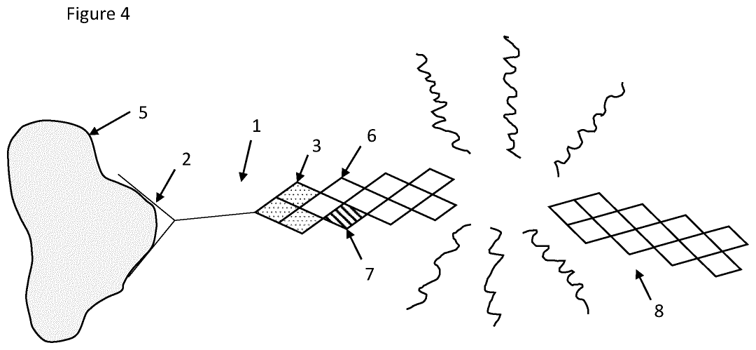

As is shown in FIG. 3, G-actin monomers 6 begin polymerising from the actin nucleus 3 of the conjugate 1 and polymerisation progresses with further G-actin monomers 6 being added to the growing polymer chain to produce an F-actin filament. In addition, labelled G-actin 7 is also incorporated into the growing polymer chain. In particular, ATP causes the G-actin monomers 6 to begin polymerising from the actin nucleus 3 on the conjugate.

ATP binds to G-actin and allows it to be bind to the F-actin strand. G-actin subunits which are bound to ATP are strongly bound to adjacent subunits in the F-actin strand. F-actin has a low rate of ATPase activity and catalyses the hydrolysis of ATP to ADP. ADP-actin subunits bind less strongly to adjacent subunits compared to ATP-actin (i.e. ADP-actin has a lower binding constant than ATP-actin). As a result, ADP-actin subunits in the filament will more readily dissociate from the filament. Therefore, even if ATP has been exhausted, ADP-actin can still undergo polymerisation, albeit, the process is less efficient due to ADP-actin's lower binding constant. As a result, to ensure efficient binding, preferably the ATP is provided in excess.

It is preferred that the G-actin monomers are stored at low temperatures to ensure the longer life of the protein and to reduce the chance of spontaneous nucleation and therefore polymerisation of the actin prior to its use with the conjugate. Also, it is desirable to store the monomers in the presence of an agent that prevents spontaneous nucleation. Tβ4 can be used for this purpose as this protein sequesters G-actin which helps to prevent spontaneous nucleation.

As noted above, actin polymerises from both the barbed plus end and the pointed minus end of F-actin filament, albeit that polymerisation that adds to the plus end of the filament is significantly faster. In the conjugate 1 of the invention, binding of the actin nucleus to the antibody does not occlude the plus or minus ends of the actin nucleus. Use of a spectrin-actin nucleus prevents polymerisation starting from the minus end of each conjugate nucleus, Additionally, adding profilin to the reaction causes profilin to associate with G-actin monomers in solution such that the G-actin monomers will only be added to the plus end of the filament and not to the minus end of the F-actin filament. Simultaneously, it helps to reduce the chance of spontaneous nucleation of the free actin.

It is quite possible to model polymerisation of actin when it can extend from both plus and minus ends of filaments, but it is simpler to model polymerisation which can only occur at a single end of the filament. Furthermore, using a single-end helps to improve the accuracy of determining the concentration of bound conjugates.

In this embodiment, the fluorescently labelled G-actin monomer 7 is pyrenyl-actin or NBD-actin (NBD: 7-chloro-4-nitrobenzeno-2-oxa-1,3-diazole). Fluorescence of these labelled G-actin monomers 7 increases when the monomer is integrated into an F-actin filament. As such, the level of fluorescence of a sample containing actin with labelled actin included therein correlates with the level of polymerisation within the sample. The higher the fluorescence, the greater the proportion of actin that is in the form of F-actin compared with G-actin.

However, labelled actin less readily associates with profilin. The result is that labelled actin sequestered less efficiently by profilin and Tβ4, and hence requires higher concentrations of profilin and Tβ4 to prevent spontaneous nucleation compared to the concentrations required to sequester unlabelled actin of identical concentration. It is for this reason that in this preferred embodiment a relatively low proportion (5%) of labelled actin is used so as to not compromise the overall polymerisation of actin in the system. A proportion of around 5 to 10% is preferred. However, this is not strictly necessary and it is nonetheless possible to use up to 100% labelled actin such that labelled actin replaces unmodified actin in the reaction.

During polymerisation, light capable of exciting the fluorescent label (such as ultravioent light) is directed at the polymerisation solution and the emitted fluorescent light from the labelled G-actin monomer 7 is detected and quantified over time, thereby providing a record of the formation of F-actin in the polymerisation solution.

After polymerisation of the F-actin filament from the conjugate 1 has progressed for a certain period of time, fragmentation of the F-actin filament spontaneously occurs, as depicted in FIG. 4. This results in the release of the conjugate 1 with a shorter filament strand and of a free actin filament 8.

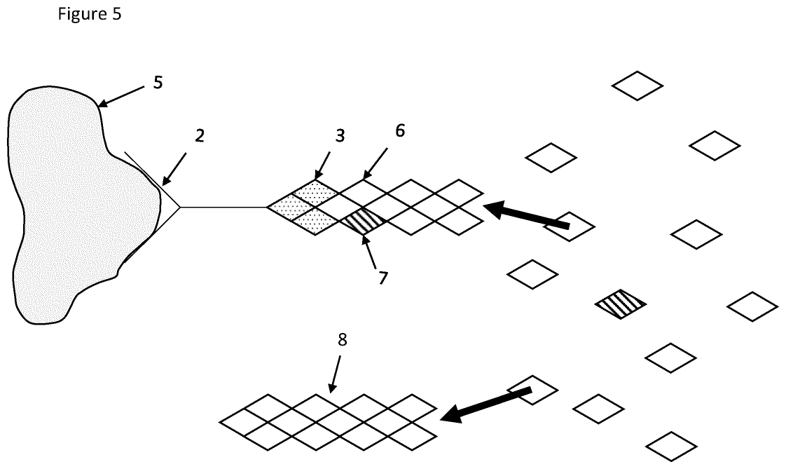

This fragmentation results in the production of a shorter filament with two free-ends from the parent filament that only had a single barbed free-end to which monomers of actin can attach. Subsequently, both the conjugate 1 and the newly-formed free filament 8 undergo further polymerisation with G-actin monomers 6,7 as shown in FIG. 5. Since there are now two free-ends, provided that G-actin is not limiting, the overall rate of polymerisation in the system is substantially doubled.

Once both the conjugate 1 and the free filament 8 have polymerised to a sufficient length, they undergo further spontaneous fragmentation resulting in the production of further free filaments 8 and therefore more free ends for polymerisation. Repeated rounds of polymerisation and fragmentation occur, after which the number of free filaments 8 resulting from fragmentation is far greater than the number of bound conjugates 1 initially provided.

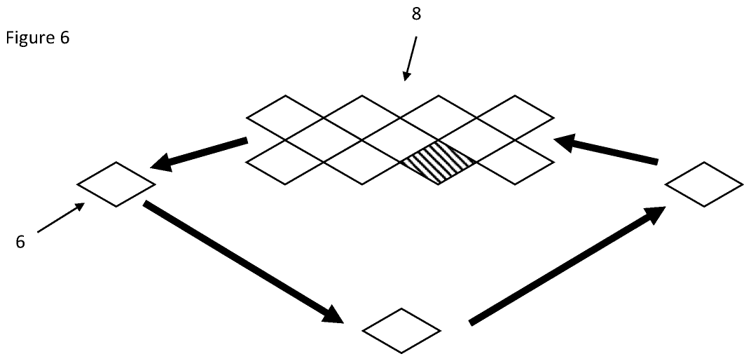

This fragmentation of filaments occurs naturally in solution as a result of the weak bonds holding together actin filaments and thermal jostling of the molecules in solution. The longer the filament, the more prone to fragmentation it becomes. As such, the natural fragmentation of actin filaments results in a gradual increase in the number of free ends at which polymerisation can occur. The overall rate of polymerisation increases until G-actin becomes limiting, at which point there is an equivalent decrease in the rate of polymerisation as the limiting concentration decreases further. As shown in FIG. 6, the actin filaments eventually reach a steady state where the rate of polymerisation of G-actin and the depolymerisation of F-actin are in steady state. In this state, G-actin is at a relatively low concentration called the critical concentration (A_{c}). The total concentration of actin (A) has not changed, simply now most of the actin is bound into filaments as F-actin and relatively little is free as G-actin. This steady state polymerisation and depolymerisation of actin filaments is called “treadmilling”. The critical concentration for ATP-actin is much lower than that for ADP-actin as ATP-actin must less readily disassociates from the filament.

Actin filaments are naturally present in solution as a polydisperse population of variable lengths as a result of the spontaneous fragmentation discussed above. Since the fragmentation does not occur at a fixed length, this complicates the determination of rate of change of rate of polymerisation as the time for a given filament to fragment is quite uncertain. Furthermore, the time taken for fragmentation is relatively long and it can take a long time for the steady state to be reached.

Therefore, in a variant of the first embodiment, the rate at which filaments undergo fragmentation is made more definite and also increased through the use of sonication. Sonication at 25 kHz is preferred but, in principle, sonication at any frequency between 1 kHz and 100 kHz is possible. Sonication causes the filaments to vibrate and fragment at more specific lengths of filament. By increasing or decreasing the frequency of the sonication applied, the length at which filaments fragment can be decreased or increased respectively. However, it is usually assumed that filaments shorter than 100 nm cannot be fragmented by increasing the frequency of sonication.

Under sonication, the length at which fragmentation occurs is shorter than that which typically occurs spontaneously due to the greater amount of energy in the system. The amount of jostling and strain that is applied to the actin filament is therefore increased. The result is that actin filaments fragment more frequently, resulting in the faster production of free-ends, therefore increasing the acceleration of polymerisation of the actin monomers such that the rate of polymerisation in the system shows a geometric increase over time.

A faster rate of change of rate of polymerisation means that the overall rate of polymerisation of actin over time is increased. This means that the time taken for G-actin to be exhausted is reduced.

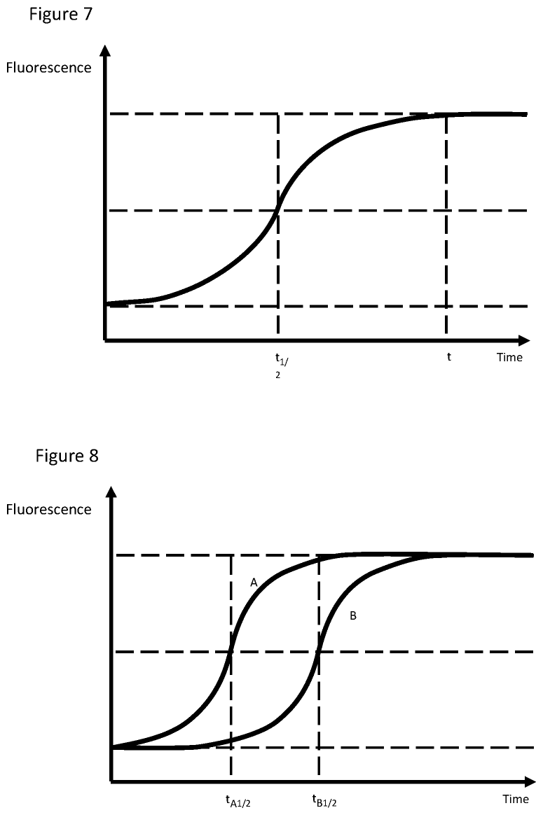

This is represented by the graph shown in FIG. 7. The graph demonstrates the polymerisation of G-actin into F-actin over time. The symmetrical geometric increase and decrease of polymerisation rate is shown through the symmetrical sigmoidal shaped line. Once the steady state is reached, the proportion of F-actin to G-actin becomes constant. This is shown in FIG. 7 where the vertical axis, which is the measured level of fluorescence (and is indicative of the total concentration of actin subtracted by the concentration of G-actin), initially increases as the concentration of G-actin decreases but then begins to plateau over time, where time is shown on the horizontal axis.

By determining the time taken to reach the steady state, and by comparing the time taken with previously-tested models, the concentration of conjugate 1 which was bound can be determined which, in turn, is indicative of the concentration of the target substance or antigen 5 in the original mixture. It is not necessary to wait for the reaction to have progressed all the way to steady state since it is possible to predict the time taken to reach the steady state at earlier points in the polymerisation process. Once, enough of the data has been measured, the remainder of the curve can be predicted. Nonetheless, waiting until steady state is reached maximises the signal obtained from the sample and would be the most accurate way to measure the time taken to reach steady state.

The amount of time taken for this steady state to be reached depends on the rate of polymerisation of actin throughout the reaction but is typically reached in a few minutes. This is shown in FIG. 8. Trace A and B represent similar polymerisation reactions but the reaction of Trace A has a higher concentration of conjugate compared to that of Trace B. Trace A reaches steady state earlier than Trace B as the initial concentration of actin nuclei at time zero in the system is higher in Trace A. As a result, the geometric increase in overall polymerisation rate takes longer to becomes apparent in the fluorescence of the reaction, As such, Trace A reaches the point of greatest incline t_{A1/2} sooner than Trace B reaches its equivalent point t_{B1/2}. It should be noted that in both cases the point of greatest incline occurs half-way between the point between the start of the reaction and the time taken to reach steady state. In both cases this is due to the concentration of G-actin beginning to become limiting.

Throughout the reaction, provided that spontaneous nucleation of the actin is minimised, the change in rate of polymerisation varies in a specific manner which can be modelled using the following equation.

$$ln ( \frac{y}{A - y} ) = \frac{k}{m} A ( t - {t}_{1 / 2} ) \qquad Equation 1$$

In Equation 1, y is a parameter equal to the total concentration of actin (A₀) subtracted by the concentration of G-actin at time t (A(t)), i.e. y=A₀−A(t). A is the concentration of actin that is available for polymerisation and is calculated by subtracting the total concentration of actin (A₀) by the critical concentration of G-actin (A_{c}) i.e. A=A₀−A_{c}. k is the sum of rate constants of actin monomer addition to filaments in polymerisation. m is the average number of actin monomer-subunits per filament, measured as an average length of filament. t_{1/2} is the time taken to reach the the polymer concentration that is half of the polymer concentration found at steady state.

The preferred point from which to base measurements is the mid-polymerisation point i.e. t_{1/2} where y(t_{1/2})=A/2. As t_{1/2} is the point of symmetry in the equation, it provides the clearest reproducible point to measure in the trace. This is because it is the point with the greatest level of polymerisation, which provides the clearest reproducible point to measure and is the point of greatest signal-to-noise ratio. Nonetheless, it is possible to measure the time taken to reach steady state from any point between the start point and the system reaching steady state albeit less accurately due to the lower signal-to-noise ratio at lower polymerisation rates.

Equation 1 can be put in terms of y to give a further equation.

$$y \vert 0 \vert = A \frac{{e}^{\frac{- k A}{m} {t}_{1 / 2}}}{1 + {e}^{\frac{- kA}{m} {t}_{1 / 2}}} \qquad Equation 2$$

In Equation 2, y|0| is the number or concentration of filaments at time zero. By measuring the time taken to reach steady state and applying Equation 2, one can then calculate the initial concentration of bound conjugates which is proportional to the concentration of the target substance to be measured.

In preferred embodiments, a particular model system is tested with known concentrations of the target substance with this method, and one can thereby determine the relative correspondence of separated conjugate concentration to the concentration of the target substance in the original sample.

This technique overcomes the issues of low signal-to-noise ratio in testing low concentrations of a target substance because the measured parameter (i.e. time taken to reach steady state) increases with decreasing concentration of the target substance.

Furthermore, the use of sonication allows for a consistent adjustable parameter that allows the time taken for the experiment to be completed to be adjusted to a preferred time frame. For example, if the concentration of the target substance is so low that the time taken to reach steady state is excessive, the frequency of sonication can be increased so as to promote faster fragmentation and therefore a faster acceleration of polymerisation, such that the time taken to reach steady state is reduced.

This method is more reliable if the conjugate 1 is provided in excess to the actual concentration of the target substance as this ensures that the maximum number of conjugates bind to the target and that no sites for binding are left unbound due to the conjugates being depleted. However, it is preferred that massive excess of the conjugate is avoided to ensure that non-specific binding of the conjugate to the target substance does not affect the amount of bound conjugate.

The amount of free actin is less important but it should still be provided in an amount that ensures significant temporal resolution between the beginning of polymerisation and the reaction reaching steady state so that samples of varying concentration have observably different times to reach steady state. Providing sufficient actin to allow for a reaction lasting between 1 to 60 minutes with the application of sonication is preferred as this allows for sufficient temporal resolution while still being fast enough to be convenient for a laboratory environment.

Kit

In one embodiment of the present invention, there is provided a kit which can be used in the method described above. The kit comprises a receptacle containing a solution of the conjugates 1 depicted in FIG. 1 or the conjugates 4 depicted in FIG. 2.

In addition, the kit comprises a receptacle containing a solution of (unlabelled) G-actin 6 and also a receptacle containing a solution of labelled G-actin 7. In some embodiments, the kit is instead provided with a single receptacle containing a mixed solution of unlabelled G-actin 6 and labelled G-actin 7 in the ratio of 19:1.

The kit is also provided with receptacles containing solutions of profilin, Tβ4,ATP, and/or actin sequestration proteins either separately or mixed together.

The kit may also be provided with receptacles containing solution buffers. These can include G-buffer (5 mM Tris.Cl pH 7.8, 0.2 mM ATP, 0.1 mM calcium chloride, 1 mM dithiothreitol, 0.01 (w/v) Sodium azide), F-buffer (G-Buffer supplemented with 0.1 M KCl and 1 mM magnesium chloride), or KME buffer (2M KCl, 20 mM magnesium chloride, 4 mM EGTA).

The kit may also be provided with a receptacle containing magnetic beads linked to an antibody complementary to the target substance. This antibody may bind to the same epitope of that of the antibody of the conjugate or alternatively it may bind to a different epitope.

Separation of the Bound Target Substance

In the first embodiment described above, bound conjugate is separated from the sample by the provision of magnetic beads and separation easing magnetic means. However, it is to be understood that such an approach is not essential to the invention.

For example, in one alternative embodiment the antigen 5 is part of a structure that falls out of solution when centrifuged, and thus centrifugation is used to obtain antigen-conjugate complexes while leaving unbound conjugates 1 in solution.

In a further embodiment, separation of bound and unbound conjugates is performed using microfluidics, filtration, adsorption to an external surface, or a combination of these methods.

Monomer

In the first embodiment described above, the monomer is G-actin which polymerises into F-actin filaments. However, it is to be understood that it is not essential to the invention that the monomer is G-actin. In principle, conjugates of the present invention may comprise a nucleus of any self-polymerising biopolymer so long as monomers of the biopolymer are provided in the polymerising solution. A “self-polymerising biopolymer” means a biologically-compatible polymer which spontaneously polymerises under predictable conditions at a geometrically increasing rate. One example of such a self-polymerising biopolymer is tubulin and, in one embodiment, the actin of the first embodiment is replaced with tubulin which polymerises into microtubules. These self-polymerising biopolymers may be dependent on an energy source to permit polymerisation, such as ATP.

Binding Element

In the first embodiment described above, the conjugate comprises an antibody which binds specifically to the target substance or antigen 5. However, it is to be understood that it is not essential to the invention that the conjugates comprises an antibody and, in principle, the conjugate may comprise any binding element which is capable of specifically binding to a target substance or antigen 5. Examples of alternative binding elements include: a lectin, a T cell receptor and a bacteriophage binding domain.

Target Substance

The present invention is not limited to any particular target substance and, in principle, the invention can relate to any target substance for which a binding element which specifically binds thereto exists. However, in preferred embodiments, the target substance is a microorganism such as a fungus, bacterium or virus.

Labelling of Monomer

In the first embodiment described above, a proportion of G-actin monomer is labelled with a fluorescent label. However, it is to be understood that fluorescent labelling, while convenient, is not essential to the invention.

For example, in one alternative embodiment, a label is not provided. Instead, the overall polymerisation of actin is determined by measurement of light scattering. Light is directed at the polymerisation solution containing actin and the light is scattered to a greater degree as the concentration of F-actin increases because the filaments disperse the light. In this way, by measuring the level of light scattering, the concentration of F-actin in the polymerisation solution is determined.

Summary of Experimental Methods and Results

Negative control of the biochemical assay: No F-actin formation was detected after extended sonication in ice-cold sonication bath for 2 hours in the following solutions. Solution 1-1.5 μM Mg⁺² G-actin (5% pyrene labelled), 2 μM profilin, 2 μM Tβ4, 5 mM Tris.Cl, pH 7.8, 2 mM MgCl₂, 0.5 mM EGTA, 0.1 M KCl. Solution 2-5 μM Mg⁺² G-actin (5% pyrene labelled), 15 μM profilin, 10 μM Tβ4, 5 mM Tris.Cl pH 7.8, 2 mM MgCl₂, 0.5 mM EGTA, 0.1 M KCl. In the absence of profilin and Tβ4, both solutions displayed polymerization in F-buffer due to spontaneous nucleation. These results indicate that spontaneous nucleation of actin can be completely prevented under polymerization conditions, by adding saturating amounts of profilin and Tβ4. Hence such a solution can serve as a ‘negative control’ for an experiment designed to assay the concentration of externally induced nuclei at time-zero of the polymerization reaction.

Nucleation experiments: VopF protein served as a nucleator for the experiments, and the assay was performed with 1 aM of VopF in solution 2. The sample was subjected to sonication in an ice-cold bath. Fluorescence emission was noted for all prior to sonication to determine the baseline. The sample was alternated manually between sonication (5 minutes) and fluorescence measurements. Steady-state polymerization was achieved within 30±5 minutes (n, 5). These results show that steady state of polymerisation can be reached in a short timeframe suitable for diagnostic purposes.

Integrating a micro-tip sonicator probe into a quartz cuvette will significantly reduce experimental error and enable a more precise determination of the t_{1/2}. It is also expected that that time to reach steady-state would be much faster if one uses an immersion sonicator-probe opposed to sonicating the sample in a bath, where F-actin fragmentation proceeds much slower due to much lower sonication energy transmitted by the bath to the sample.

Bacterial quantification experiments through actin nucleation kinetics: Streptavidin coated magnetic nanoparticles (Mp) (0.1-1.0 μm) saturated with biotinylated polyclonal antibody raised against multiple K and O strains of E. coli, were incubated for 30 minutes with E. coli TB1 strain (resistant to streptomycin), at a bacterial concentration of 10 CFU/ml, along with latex nanoparticles (250 nm) that were crosslinked with polyclonal antibody and VopF. The sample (10 ml) was incubated with 100 mg each of Mps and latex nanoparticles. Subsequently, the Mps were collected using a magnet and washed thrice with 1×PBS. After the washes, the Mps were reconstituted in 300 μl of solution 2, and the sample was subjected to sonication in an ice-cold bath. The sonication was interrupted every 10 minutes, and the solution was assayed for F-actin content. The sonication-time required to reach steady state in five separate attempts for detecting 10 CFU/ml was 50±20 min. Optimizing the nanoparticle-protein crosslinking chemistry of Mp and latex particles is likely to improve the sensitivity of the assay. Detection of bacteria in samples with concentrations <10 CFU/ml are yet to performed.

1. A conjugate for measuring the amount of a target substance comprising:
a. a binding element capable of binding to the substance
b. a self-polymerising biopolymer or a nucleating protein.

2. The conjugate according to claim 1, wherein the binding element is an antibody, a lectin, a T-cell receptor, a bacteriophage binding domain, an opsonin, a lectin, or a synthetic ligand, preferably wherein the synthetic ligand is zinc-coordinated bis(dipicolylamine) for binding selectively to bacterial cell walls.

3. The conjugate according to claim 1, wherein the biopolymer is in the form of a self-polymerising biopolymer nucleus for seeding biopolymer polymerisation.

4. The conjugate according to claim 1, wherein the biopolymer is actin or tubulin.

5. The conjugate according to claim 1, wherein the conjugate further comprises a nanoparticle to which the binding element and the biopolymer are linked.

6. A kit comprising:
a. the conjugate according to claim 1;
b. a plurality of self-polymerising biopolymer subunits.

7. The kit according to claim 6, further comprising an agent to prevent spontaneous nucleation or polymerisation of the biopolymer subunits.

8. The kit according to claim 6, wherein the biopolymer subunits comprises labelled self-polymerising biopolymer subunits.

9. A method for measuring the amount of a target substance, the method comprising:
a. providing a plurality of conjugates as defined in claim 1;
b. binding the conjugates to the target substance;
c. isolating bound conjugates;
d. polymerising biopolymer filaments;
e. calculating an amount of the target substance.

10. The method according to claim 9, wherein the method further comprises the additional step:
d2. measuring a time value indicative of the time taken for polymerisation and depolymerisation of the biopolymer to reach steady state.

11. The method according to claim 17 wherein calculation in step e comprises using the calculated number or concentration of bound conjugates to calculate the amount of the target substance.

12. The method according to claim 9, wherein step d further comprises increasing the rate at which biopolymer filaments undergo fragmentation, preferably by using sonication.

13. The method according to claim 12, wherein a number or concentration of bound conjugates is calculated using the following equation:
$$y \vert 0 \vert = A \frac{{e}^{\frac{- k A}{m} {t}_{1 / 2}}}{1 + {e}^{\frac{- kA}{m} {t}_{1 / 2}}}$$
where y|0| is the concentration of bound conjugates at time zero,
where A is the total concentration of biopolymer minus the critical concentration of biopolymer,
where k is the sum of rate constants of biopolymer subunit addition to filament ends,
where m is the average length of the biopolymer filaments
and where t_{1/2} is the time taken to reach the biopolymer concentration that is half of the biopolymer concentration found at steady state.

14. The method according to claim 10, wherein the polymerisation state of the biopolymer is measured by using fluorescently-labelled biopolymer.

15. The method according to claim 10, wherein the polymerisation state of the biopolymer is measured by the level of light scattering caused by the biopolymer filaments.

16. The conjugate according to claim 1, wherein the biopolymer is tubulin.

17. The method according to claim 10, wherein the method further comprises the additional step:
d3. using the time value to calculate a number or concentration of the bound conjugates.
